(19) United States
(12) Patent Application Publication
(10) Pub. No.: US 2004/0024215 A1
(43) Pub. Date: Feb. 5, 2004

(54) Benzamide derivatives and their use as apob-100 and mtp inhibitors

(76) Inventors: Alain Claude - Marie Daugan, Les Ulis (FR); Nerina Dodic, Les Ulis (FR)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/296,681

(22) PCT Filed: Jun. 1, 2001
(86) PCT No.: PCT/EP01/06244

(30) Foreign Application Priority Data
Jun. 1, 2000 (GB) 0013346.2

(51) Int. Cl. C 07D 4 3/02; C07D 241/04

(52) U.S. Cl. 544/360; 544/372; 544/392

(57) ABSTRACT

The present invention relates to a compound of formula (I); wherein R¹ represents isopropyl or trifluoromethyl; R² represents hydrogen, C₁₋₄alkyl, chloro, fluoro or trifluoromethyl; R³ represents(i) phenyl, optionally substituted by cyano, halogen, trifluoromethyl or an optionally substituted 5-membered heteroaromatic group, where optional substitution is effected by C₁₋₄alkyl, (ii) a 5-membered heteroaromatic group, optionally substituted by halogen, cyano or C₁₋₄alkyl, (ii) aminocabonyl, or(iv) ethyl or eth-1-enyl; R⁴ represents cyano, methyl, acetyl, a 5-membered heteoaromatic group, optionally substituted by C₁₋₄alkyl or phenyl or a group X—Y-Z; X represents a carboxy, oxo, C₁₋₆alkylene, carboxamido or thiocarboxamido linking group; Y represents a direct link or C₁₋₄alkylene, Z represents (i) hydrogen, (ii) trifluoromethyl, (iii) cyano, (iv) phenyl (v) a 5- or 6-membered heteroaromatic group, optionally substituted by C₁₋₄alkyl, with the proviso that when X represents C₁₋₄alkylene, Y and Z do not represent a direct link and hydrogen respectively, or when X represents oxo, Y and Z do not represent C₁₋₆alkylene and hydrogen respectively; or a physiologically acceptable salt, solvate or derivative thereof, to compositions comprising the compound, processes for their preparation and their use in treating conditions ameliorated by an apoB-100 and/or MTP inhibitor.

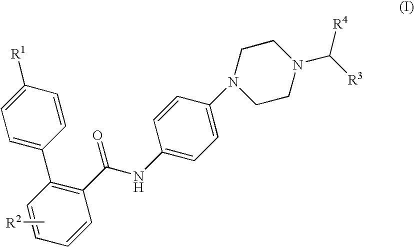

The invention relates to therapeutic benzamide derivatives, their use in inhibiting hepatic production of apoprotein B-100 (apoB-100) and intestinal production of chylomicrons or apoprotein B-48 (apoB-48) and MTP, and intermediates useful in the production of such derivatives.

ApoB-100 is the main protein component of low density lipoprotein-cholesterol (LDL-c). High LDL-c plasmatic levels are a major risk factor for atherosclerosis and coronary artery diseases. ApoB-48 is the main protein component of chylomicrons.

The microsomal triglyceride transfer protein (MTP) catalyses the transfer of triglycerides, cholesteryl esters and phosphatidylcholine between small unilamellar vesicles. MTP is expressed in liver and intestine, both organs which produce lipoproteins. MTP is able to lipidate neosynthesized apoB-100 within the liver, and neosynthesized apoB-48 within the intestine, therefore leading to the production of triglyceride-rich lipoparticles such as VLDL and chylomicrons respectively. Thus, MTP inhibitors have the potential to decrease LDL-c and triglyceride plasmatic levels, and also intestinal lipid absorption. MTP inhibitors may be used in the treatment of non-insulin dependent diabetes mellitus, coronary heart disease, pancreatitis, mixed dyslipidemia, hypercholesterolemia, hypertriglyceridemia, hyperlipemia, post-prandial hyperlipemia, atherosclerosis and obesity.

Compounds having apoB-100 and MTP inhibition properties have been described in WO96/40640. International Patent Application no. PCT/EP99/09320 describes therapeutic benzamide compounds for the treatment of conditions resulting from elevated circulating levels of apoB-100.

Thus, the present invention provides a compound of formula (I);

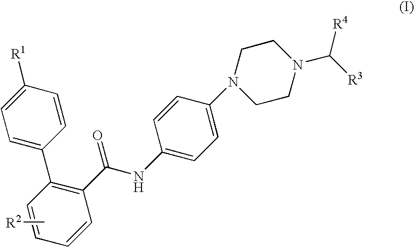

wherein

R¹ represents isopropyl or trifluoromethyl;

R² represents hydrogen, C₁₋₄alkyl, chloro, fluoro or trifluoromethyl;

R³ represents

(i) phenyl, optionally substituted by cyano, halogen, trifluoromethyl or an optionally substituted 5-membered heteroaromatic group, where optional substitution is effected by C₁₋₄alkyl, or

(ii) a 5-membered heteroaromatic group, optionally substituted by halogen, cyano or C₁₋₄alkyl,

(iii) aminocarbonyl, or

(iv) ethyl or eth-2-enyl;

R⁴ represents cyano, methyl, acetyl, a 5-membered heteroaromatic group, optionally substituted by C₁₋₄alkyl or phenyl, or a group X—Y-Z;

X represents a carboxy, oxo, C₁₋₆alkylene, carboxamido or thiocarboxamido linking group;

Y represents a direct link or C₁₋₆alkylene;

Z represents

(i) hydrogen,

(ii) trifluoromethyl,

(iii) cyano,

(iv) phenyl,

(v) a 5- or 6-membered heteroaromatic group, optionally substituted by C₁₋₄alkyl,

with the proviso that when X represents C₁₋₆alkylene, Y and Z do not represent a direct link and hydrogen respectively, or when X represents oxo, Y and Z do not represent C₁₋₆alkylene and hydrogen respectively;

or a physiologically acceptable salt, solvate or derivative thereof.

Suitable physiologically acceptable salts of the compounds of general formula (I) include acid addition salts formed with pharmaceutically acceptable organic and inorganic acids for example, citrates, hydrochlorides, hydrobromides, or sulphates. Particularly preferred salts are citrates or hydrochloride salts. The solvates may, for example, be hydrates.

References hereinafter to a compound according to the invention include both compounds of formula (I) and their physiologically acceptable salts together with physiologically acceptable solvates.

Referring to the general formula (I), alkyl, alkylene and alkoxy include both straight and branched chain saturated hydrocarbon groups. Examples of alkyl groups include methyl and ethyl groups, examples of alkylene groups include methylene and ethylene groups, whilst examples of alkoxy groups include methoxy and ethoxy groups.

Referring to the general formula (I), eth-2-enyl refers to a ethyl group comprising one double bomnd, where the double bond is adjacent the linking group rather than at the terminal group.

Referring to the general formula (I), reference to a heteroaromatic group, unless otherwise defined, means any single aromatic ring containing at least one ring heteroatom independently selected from O, N and S.

Referring to the general formula (I), reference to a halogen group includes fluoro, chloro, bromo and iodo groups.

R¹ is preferably isopropyl.

R² is suitably isopropyl or trifluoromethyl. R² is preferably methyl or isopropyl, most preferably methyl. R² is suitably 5- or 6-substituted, preferably 6-substituted.

R³ is suitably selected from phenyl, optionally substituted by cyano, trifluoromethyl or halogen, e.g. bromo or fluoro, or a 5-membered heteroaromatic group, e.g. 2-pyrrolyl. Where R³ is an optionally substituted phenyl, the substituent is suitably in the 3- or 4-position, preferably the 3-position.

R⁴ suitably represents

(i) cyano,

(ii) benzoyl,

(iii) hydroxycarbonyl, C₁₋₄alkoxycarbonyl, e.g. methoxycarbonyl, C₁₋₃perfluoroalkylaminocarbonyl, e.g. 1,1,1-trifluoroethylaminocarbonyl,

(iv) aminothiocarbonyl;

(v) a 5-membered heteroaromatic group, e.g. oxadiazolyl or pyrrolyl, optionally substituted by phenyl, or

(vi) a 5-membered heteroaromatic group linked by a methylene, e.g. pyrazolylmethyl.

Particularly preferred compounds of the invention include those in which each variable in formula (I) is selected from the preferred groups for each variable. Even more preferable compounds of the invention include, those where each variable in formula (I) is selected from the more preferred or most preferred groups for each variable.

A suitable sub-group of a compound of formula (I) is represented by a compound of formula (Ia)

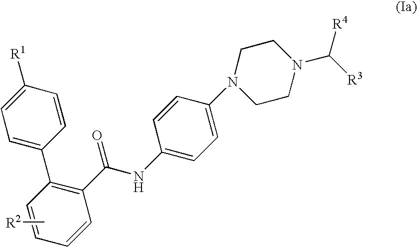

wherein

R¹ represents isopropyl or trifluoromethyl;

R² represents hydrogen, C₁₋₄alkyl, chloro, fluoro or trifluoromethyl;

R³ represents

(i) phenyl, optionally substituted by cyano, halogen, trifluoromethyl or an optionally substituted 5-membered heteroaromatic group, where optional-substitution is effected by C₁₋₄alkyl, or

(ii) a 5- membered heteroaromatic group, optionally substituted by halogen, cyano or C₁₋₄alkyl;

R⁴ represents cyano, a 5-membered heteroaromatic group, optionally substituted by C₁₋₄alkyl or phenyl, or a group X—Y-Z;

X represents a carboxy, oxo, C₁₋₆alkylene, carboxamido or thiocarboxamido linking group;

Y represents a direct link or C₁₋₆alkylene;

Z represents

(i) hydrogen,

(ii) trifluoromethyl,

(iii) cyano,

(iv) phenyl,

(v) a 5- or 6-membered heteroaromatic group, optionally substituted by C₁₋₄alkyl,

with the proviso that when X represents C₁₋₆alkylene, Y and Z do not represent a direct link and hydrogen respectively,- or when X represents oxo, Y and Z do not represent C₁₋₆alkylene and hydrogen respectively;

or a physiologically acceptable salt, solvate or derivative thereof.

It will be clear that references herein to a compound of formula (I) apply equally to a compound of formula (Ia).

Suitable compounds according to the invention include:

4′-trifluoromethyl-biphenyl-2-carboxylic acid[4-(4-(α-methyl-benzyl)-piperazin-1-yl)-phenyl]-amide;

5-methyl-4′-trifluoromethyl-biphenyl-2-carboxylic acid[4-(4-(3-cyano-α-methyl-benzyl)-piperazin-1-yl)-phenyl]-amide;

4′-isopropyl-6-methyl-biphenyl-2-carboxylic acid[4-(4-(3-cyano-α-methyl-benzyl)-piperazin-1-yl)-phenyl]-amide;

4′-trifluoromethyl-biphenyl-2-carboxylic acid[4-(4-(3-trifluoromethyl-α-methyl-benzyl)-piperazin-1-yl)-phenyl]-amide;

4′-trifluoromethyl-biphenyl-2-carboxylic acid[4(4-(α-cyano-benzyl)-piperazin-1-yl)-phenyl]-amide;

4′-trifluoromethyl-biphenyl-2-carboxylic acid[4-(4-(3-bromo-α-cyano-benzyl)-piperazin-1-yl)-phenyl]-amide;

4′-trifluoromethyl-biphenyl-2-carboxylic acid[4-(4-(4-fluoro-α-acetyl-benzyl)-piperazin-1-yl)-phenyl]-amide;

4′-trifluoromethyl-biphenyl-2-carboxylic acid[4-(4-(α-benzoyl-benzyl)-piperazin-1-yl)-phenyl]-amide;

4′-trifluoromethyl-biphenyl-2-carboxylic acid[4-(4-(α-(5-phenyl-[1,2,4]oxadiazol-3-yl)-benzyl)-piperazin-1-yl)-phenyl]-amide;

4′-trifluoromethyl-biphenyl-2-carboxylic acid[4-(4(α-((pyrazol-1-yl)-methyl-benzyl)-piperazin-1-yl)-phenyl]-amide;

4′-trifluoromethyl-biphenyl-2-carboxylic acid[4-(4-(α-(methoxycarbonyl)-benzyl)-piperazin-1-yl)-phenyl]-amide;

4′-trifluoromethyl-biphenyl-2-carboxylic acid[4-(4-(α-(carboxy)-benzyl)-piperazin-1-yl)-phenyl]-amide;

4′-trifluoromethyl-biphenyl-2-carboxylic acid[4-(4-(α-((2,2,2-trifluoroethyl)-aminocarbonyl)-benzyl)-piperazin-1-yl)-phenyl]-amide;

4′-trifluoromethyl-biphenyl-2-carboxylic acid[4-(4-(α-((pyridin-2-yl-methyl)-aminocarbonyl)-benzyl)-piperazin-1-yl)-phenyl]-amide;

4′-trifluoromethyl-biphenyl-2-carboxylic acid[4-(4-(3-bromo-α-thiocarbamoyl-benzyl)-piperazin-1-yl)-phenyl]-amide;

4′-trifluoromethyl-biphenyl-2-carboxylic acid[4-((4(3-bromo-α-(4-methyl-thiazol-2-yl)-benzyl)-piperazin-1 -yl)-phenyl]-amide;

4′-trifluoromethyl-biphenyl-2-carboxylic acid[4-(4(3-cyano-α-(pyrrol-2-yl)-benzyl)-piperazin-1-yl)-phenyl]-amide;

4′-trifluoromethyl-biphenyl-2-carboxylic acid[4-(4(-α-methyl-pyrrol-2-yl)-piperazin-1-yl)-phenyl]-amide;

or a physiologically acceptable salt, solvate or derivative thereof.

The term “physiologically functional derivative” as used herein refers to any physiologically acceptable derivative of a compound of the present invention, for example, an ester or amide, which upon administration to a mammal, such as a human, is capable of providing (directly or indirectly) such a compound or an active metabolite thereof. Such derivatives are clear to those skilled in the art, without undue experimentation, and with reference to the teaching of Burger's Medicinal Chemistry And Drug Discovery, 5th Edition, Vol 1: Principles And Practice, which is incorporated herein by reference.

The compounds of the invention are inhibitors of hepatic production of apoB-100 and MTP and are thus of use in the treatment of conditions ameliorated by an apoB-100 and/or MTP inhibitor.

The ability of the compounds of this invention to inhibit human MTP activity is measured by an in vitro assay where MTP tranfers 3H-triolein between phosphatidylcholine liposomes. The specificity of the compounds of the invention is established by comparing the effects on apoB-100 and apoprotein A-1 production. A specificity of at least 100 is preferred.

The in vivo profile of the compounds is determined by acute oral administration of the compounds of the invention to DBA/2 mice and Wistar rats. Potency of the active compounds is evaluated by measuring plasmatic lipids (total cholesterol, triglyceride, LDL cholesterol and HDL cholesterol) and apoproteins (apoB-100, apoB-48 and apoA-1).

The compounds of the invention are potent and specific inhibitors of hepatic production of apoB-100 and MTP, which furthermore exhibit good oral bioavailability and duration of action.

Compounds of the invention are of use in the treatment of atherosclerosis, pancreatitis, non-insulin dependent diabetes mellitus (NIDDM), coronary heart diseases and obesity.

Compounds of the invention are also useful in lowering serum lipid levels, cholesterol and/or triglycerides, and are of use in the treatment of hyperlipemia, hyperlipidemia, post-prandial hyperlipemia, mixed dislipidemia, hyperlipoproteinemia, hypercholesterolemia and/or hypertriglyceridemia.

The invention therefore provides a compound of formula (I) or a physiologically acceptable salt, solvate or derivative thereof for use in therapy, in particular in human medicine.

There is also provided as a further aspect of the invention the use of a compound of formula (I) or a physiologically acceptable salt, solvate or derivative thereof in the preparation of a medicament for use in the treatment of conditions ameliorated by an apoB-100 and/or MTP inhibitor.

In an alternative or further aspect, there is provided a method for the treatment of a mammal, including man, comprising administration of an effective amount of a compound of formula (I) or a physiologically acceptable salt, solvate or derivative thereof in particular in the treatment of conditions ameliorated by an apoB-100 and/or MTP inhibitor.

It will be appreciated that reference to treatment is intended to include prophylaxis as well as the alleviation of established symptoms. Compounds of formula (I) may be administered as the raw chemical but the active ingredient is preferably presented as a pharmaceutical formulation.

Accordingly, the invention also provides a pharmaceutical composition which comprises at least one compound of formula (I) or a physiologically acceptable salt, solvate or derivative thereof and formulated for administration by any convenient route. Such compositions are preferably in a form adapted for use in medicine, in particular human medicine, and can conveniently be formulated in a conventional manner using one or more pharmaceutically acceptable carriers or excipients.

Thus compounds of formula (I) may be formulated for oral, buccal, parenteral, transdermal, topical (including ophthalmic and nasal), depot or rectal administration or in a form suitable for administration by inhalation or insufflation (either through the mouth or nose).

The compounds of formula (I) may, if desired, be administered with one or more therapeutic agents and formulated for administration by any convenient route in a conventional mann r. Appropriate doses will be readily appreciated by those skilled in the art. For example, the compounds of formula (I) may be administered in combination with an HMG CoA reductase inhibitor.

A compound of formula (I), or a physiologically acceptable salt, solvate or derivative thereof, may be prepared by the general methods outlined hereafter. In the following description, the groups R¹, R², R³ and R⁴ are as previously defined for compounds of formula (I), unless specified otherwise.

According to a general process (A), a compound of formula (I) may be prepared by reacting a compound of formula (II) with a compound of formula R³(R⁴)L

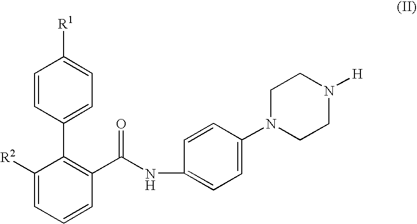

where L represents a suitable leaving group, e.g. a halide such as chloride, or a hydroxy group under standard displacement conditions.

A compound of formula (II) may be prepared by reaction of a compound of formula (Ill) with a compound of formula (IV)

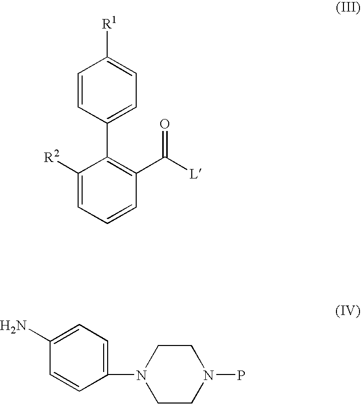

where L′ is a suitable leaving group, such as chloride, or an OH group and P is a suitable amine protecting group, e.g. tert-butoxycarbonyl (Boc), under standard coupling conditions for an acid and amine coupling, followed by deprotection of the protecting group under suitable conditions, e.g. acidic removal of a Boc group.

A compound of formula (1V) may be prepared by the two step reaction of a compound of formula (V)

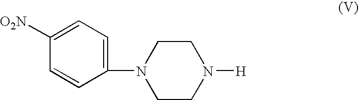

comprising incorporation of the protecting group P using standard methodology followed by reduction of the nitro group, e.g. under hydrogenation conditions.

According to a second method (B), compounds of formula (I) may be prepared by reaction of compounds of formula (Ill) and compounds of formula (VI)

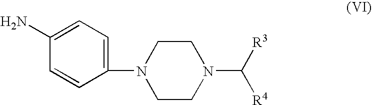

where L is defined above, under standard coupling conditions.

Compounds of formula (VI) may be prepared by reaction of a compound of formula () with a compound of formula R³-L, where L is defined above, followed by reduction of the nitro group under hydrogenation or reductive tin chloride conditions.

According to a third general process (C), a compound of formula (I), where there is an alkylene link to the piperidine or piperazine group, may be prepared by reacting a compound of formula (II) with a compound of formula (VII)

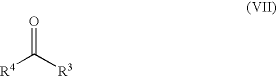

under standard reductive amination conditions, e.g. using sodium triacetoxyborohydride in a solvent such as dichloroethane.

According to a fourth process (D), a compound of formula (I) may be prepared from a different compound of formula (I), using standard techniques well known in the art. For example, compounds of formula (I) where R⁴ comprises a group containing an amide group may be prepared from the compound of formula (I) where the corresponding position comprises a carboxylic acid group, which in turn may be prepared from the compound of formula (I) where the corresponding position comprises a carboxylic ester group. Well known methods in the art may be employed to facilitate the transformation of an ester to an acid and then to an amide.

A compound of formula (III), where L′ is a hydroxy group, may be prepared firstly by coupling a boronic acid with a suitable leaving group, represented by a compound of formula (VIII) and a compound of formula (IX)

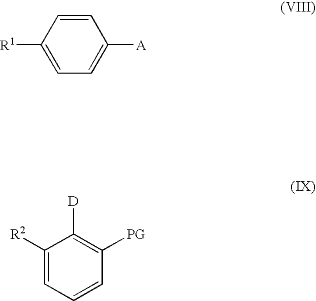

where PG represents a protected carboxylic acid and A and D represent either the boronic acid or the suitable leaving group, such as triflate or bromide, followed by deprotection of the protecting group under standard conditions, such as base removal of an ester group. Where L represents a halide leaving group, the carboxylic acid product can be treated with a suitable reagent, such as thionyl chloride, to give the corresponding chloride leaving group.

Where R³ is a phenylmethyl, substituted by an aromatic heterocyclyl, the aromatic heterocyclyl may be introduced by any well known methods in the art. For instance, where the substituent is a methyl substituted oxadiazole, this may be formed by treatment of a suitable benzamide derivative with a suitable reagent, such as dimethylacetamide dimethylacetal at elevated temperature, followed by cyclisation of the intermediate compound with hydoxylamine.

The various general methods described above may be useful for the introduction of the desired groups at any stage in the stepwise formation of the required compound, and it will be appreciated that these general methods can be combined in different ways in such multi-stage processes. The sequence of the reactions in multi-stage processes should of course be chosen so that the reaction conditions used do not affect groups in the molecule which are desired in the final product.

Compounds of formula R³(R⁴)L, (III), (V), (VII), (VIII) and (IX) are known or may be prepared by standard methods well known in the art and/or herein described.

Physiologically acceptable salts may also be prepared from other salts, including other physiologically acceptable salts, of the compound of formula (I) using conventional methods.

The compounds of formula (I) may readily be isolated in association with solvent molecules by crystallisation from or evaporation of an appropriate solvent to give the corresponding solvates.

When a specific enantiomer of a compound of general formula (I) is required, this may be obtained for example by resolution of a corresponding enantiomeric mixture of a compound of formula (I) using conventional methods.

Thus, in one example an appropriate optically active acid may be used to form salts with the enantiomeric mixture of a compound of general formula (I). The resulting mixture of isomeric salts may be separated, for example, by fractional crystallisation into the diastereoisomeric salts from which the required enantiomer of a compound of general formula (I) may be isolated by conversion into the required free base.

Alternatively, enantiomers of a compound of general formula (I) may be synthesised from the appropriate optically active intermediates using any of the general processes described herein.

The invention is further illustrated by the following intermediates and examples. All temperatures are in degrees centigrade.

Abbreviations:

MS-LCMS mass spectrography, HOBt-1-Hydroxybenzotriazole, AcOEt-Ethyl acetate, EDCl-1-(3dimethylaminopropyl)-3-ethylcarbodiimide hydrochloride, BINAP-2,2′-bis(diphenylphosphino)-1,1′-binaphthyl, THF-Tetrahydrofuran, MeOH-Methanol, EtOH-Ethanol, Et₃N-Triethylamine

Intermediate 1

4′-6diisopropyl-biphenyl-2-carboxylic acid methyl ester

To a stirred solution of 3-isopropyl-2-(trifluoro-methanesulfonyloxy)-benzoic acid methyl ester (2.3 g) in toluene (15 ml) was added LiCl (0.88 g) and Pd(PPh3)4 (0.402 g. After 10 minutes at room temperature, a 2M solution of Na2CO3 (7 ml) was added followed by 4-isopropylphenyl boronic acid (1.43 g) in EtOH (10 ml). The resulting mixture was heated under reflux during 6 hours and then cooled to room temperature. After decantation, the organic phase was diluted, washed with water, dried over Na2SO4, filtered and concentrated under reduced pressure. The titled compound was obtained as a brown oil (2.07 g)

GC/MS: m/z 296 (M+)

Similarly prepared:

Intermediate 2

4′-trifluoromethyl-6-isopropyl-biphenyl-2-carboxylic acid methyl ester as an oil which crystallised (2.25 g)

GC/MS: m/z 322 (M+)

from of 3-isopropyl-2-(trifluoro-methanesulfonyloxy)-benzoic acid methyl ester (2.3 g) and 4-trifluoromethylphenyl boronic acid (1.59 g)

Intermediate 3

6-fluoro-4′-isopropyl-biphenyl-2-carboxylic acid methyl ester

as an oil (1.7 g)

GC/MS: m/z 272 (M+)

from of 3-fluoro-2-(trifluoro-methanesulfonyloxy)-benzoic acid methyl ester (2.08 g) and 4-isopropylphenyl boronic acid (1.27 g)

Intermediate 4

6-fluoro4′-trifluoromethyl-biphenyl-2-carboxylic acid methyl ester

as an oil (1.9 g)

GC/MS: m/z 298 (M+)

from of 3-fluoro-2-(trifluoro-methanesulfonyloxy)-benzoic acid methyl ester (2.08 g) and 4-trifluoromethylphenyl boronic acid (1.48 g)

Intermediate 5

6-chloro-4′-isopropyl-biphenyl-2-carboxylic acid methyl ester as an dark oil (3 g)

GC/MS: m/z 288 (M+).

from of 3-chloro-2-(trifluoro-methanesulfonyloxy)-benzoic acid methyl ester (3.5 g) and 4-isopropylphenyl boronic acid (2.28 g)

Intermediate 6

6-chloro4′-trifluoromethyl-biphenyl-2-carboxylic acid methyl ester

as an oil (g)

from of 3-chloro-2-(trifluoro-methanesulfonyloxy)-benzoic acid methyl ester (g) and 4-trifluoromethylphenyl boronic acid (g)

Intermediate 7

4′-isopropyl-4-trifluoromethyl-biphenyl-2-carboxylic acid methyl ester

To a mixture of NiCl2(dppf) (0.5 g) in dioxane (30 ml) was added dropwise BuLi (solution 2M in cyclohexane, 1.5 ml) and the mixture was stirred at room temperature during 10 minutes. Then were added 4-isopropylphenyl boronic acid (1.43 g), K3PO4 (4.65 g) and 2-chloro3-trifluoromethyl-benzoic acid methyl ester (1.7 g) and the mixture was heated under reflux overnight. The catalyst was filtered off and the filtrate concentrated under reduced pressure. The residue was treated with water, extracted with diethyle oxyde. The organic phase was washed with water, dried over Na2SO4 and concentrated. After purification by flash chromatography eluting with cyclohexane/AcOEt (92/8), the titled compound was obtained as an oil (0.37 g)

GC/MS: m/z 322 (M+)

Intermediate 8

4′-6-diisopropyl-biphenyl-2-carboxylic acid To a Stirred Solution of 4′-6-diisopropyl-biphenyl-2-carboxylic acid methyl ester (2.07 g) in ethanol (10 ml) was added NaOH (solution 1N, 21 ml) and the mixture was heated under reflux overnight. After concentration under reduced pressure, the residue was taken in water and the aquous phase was washed with diethyle oxyde and then made acidic with HCl (solution 1N). The aquous phase was extracted with diethyle oxyde and the organic phase was dried over Na2SO4, filtered and concentrated under reduced pressure. After crystallisation from MeOH/H2O, the titled compound was obtained as white crystals (1.6 g)

Mp:123-125° C.

Similarly prepared:

Intermediate 9

6-isopropyl-4′-trifluoromethyl-biphenyl-2-carboxylic acid

as white crystals (1.5 g)

mp:178-180° C.

from 6-isopropyl-4′-trifluoromethyl-biphenyl-2-carboxylic acid methyl ester (2.25 g)

Intermediate 10

6-fluoro4′-isopropyl-biphenyl-2carboxylic acid

as white crystals (1.6 g)

mp:125-127° C.

from 6-fluoro-4′-isopropyl-biphenyl-2-carboxylic acid methyl ester (1.7 g)

Intermediate 11

6-fluoro-4′-trifluoromethyl-biphenyl-2-carboxylic acid

as white crystals (1.5 g)

mp:185-187° C.

from 6-fluoro4′-trifluoromethyl-biphenyl-2-carboxylic acid methyl ester (1.9 g)

Intermediate 12

6-chloro-4′-isopropyl-biphenyl-2-carboxylic acid

as a white solid (2.3 g)

mp:106-108° C.

from 6-chloro-4′-isopropyl-biphenyl-2-carboxylic acid methyl ester (3 g)

Intermediate 13

6-chloro-4′-trifluoromethyl-biphenyl-2-carboxylic acid

as white solid (g)

mp:° C.

from 6-chloro4′-trifluoromethyl-biphenyl-2-carboxylic acid methyl ester (g)

Intermediate 14

4′-isopropyl-6-trifluoromethyl-biphenyl-2-carboxylic acid

as a white solid (0.3 g)

mp:111-113° C.

from 4′-isopropyl-6-trifluoromethyl-biphenyl-2-carboxylic acid methyl ester (0.37 g)

General Method for Acid/Amine Couplings of Intermediates 15-16

To a stirred solution of the 1-benzyl-piperazine aniline, 4′-isopropyl-6-methyl-biphenyl-2-carboxylic acid (1eq), HOBT (1 eq) and triethylamine (at least 1 eq) in CH2Cl2 (50 ml) was added EDCl (1 eq) and the mixture was heated at 40° C. overnight. The mixture was diluted with CH2Cl2, and the organic solution was washed with water, then with a saturated solution of NaHCO3, then with a saturated solution of NaCl and dried over Na2SO4. After filtration and evaporation of the filtrate, the residue was purified by flash chromatography eluting with AcOEt/CH2Cl2 (50/50) to give the titled compound

Intermediate 15

4′,6-diisopropyl-biphenyl-2-carboxylic acid [4-(4-terbutyloxycarbonyl-piperazin-1-yl)-phenyl]-amide

as a cream powder (27 g)

mp: 158-160° C.

from 4′,6-diisopropyl-biphenyl-2-carboxylic acid (14.25 g) and 1-terbutyloxycarbonyl-4-(4-aminophenyl)-piperazine (14 g)

Intermediate 16

6-isopropyl-4′-trifluoromethyl-biphenyl-2-carboxylic acid [4-(4-terbutyloxycarbonyl-piperazin-1-yl)-phenyl 1-amide

as a powder (1.25 g)

mp: 100° C.

from 6-isopropyl-4′-trifluoromethyl-biphenyl-2-carboxylic acid (1 g) and 1 -terbutyloxycarbonyl-4-(4-aminophenyl)-piperazine (0.9 g)

General Method for Removal of 1-benzyl Group from piperazines, Intermediates 17-18

A solution of 4′-isopropyl-6-methyl-biphenyl-2-carboxylic acid [2-(4-benzyl-piperazin-1-yl)-pyridin-5-yl]-amide in EtOH (200 ml) and CH2Cl2 (10 ml) containing Pd/C was hydrogenated at room temperature. After 24 hours, the catalyst was removed by filtration and the filtrate was evaporated under reduced pressure to give the titled compound.

Similarly prepared:

Intermediate 17

4′,6-diisopronyl-biphenyl-2-carboxylic acid [4-(piperazinyl)-phenyl]-amide

as a white powder (19 g)

from 4′,6-diisopropyl-biphenyl-2-carboxylic acid [4-(4-terbutyloxycarbonyl-piperazin-1-yl)-phenyl]-amide (27 g)

Intermediate 18

6-isopropyl-4′-trifluoromethyl-biphenyl-2-carboxylic acid [4-(piperazinyl)-phenyl]-amide

as a white powder (1 g)

mp: 203-205° C.

from 6-isopropyl-4′-trifluoromethyl-biphenyl-2-carboxylic acid [4-(4-terbutyloxycarbonyl-piperazin-1-yl)-phenyl]-amide (1.25 g)

### EXAMPLE 1

4′-Trifluoromethyl-biphenyl-2-carboxylic acid [4-(4-(α-methyl-benzyl)-piperazin-1-yl)-phenyl]-amide

To a stirred solution of 4′-trifluoromethyl-biphenyl-2-carboxylic acid [4-(piperazinyl)-phenyl]-amide (318 mg) in acetone (10 mL) were added potassium carbonate (228 mg) and then α-methylbenzyl bromide (152 mg) and the mixture was heated under reflux during 16 hours. The salts were filtered off and the filtrate was concentrated under reduced pressure. The residue was purified by flash chromatography eluting with CH₂Cl₂/MeOH (95/5) to give a solid which was recrystallised from EtOH. The title compound was obtained as white crystals (0.35 g).

m.p.: 194-196° C.

MS: m/z 530 (M+1).

Similarly prepared were:

### EXAMPLE 2

5-Methyl-4′-trifluoromethyl-biphenyl-2-carboxylic acid [4-(4-(3-cyano-α-methyl-benzyl)-piperazin-1-yl)-phenyl]-amide as white crystals (30 mg),

m.p.: 192-194° C.

MS: m/z 569 (M+1)

from 5-methyl-4′-trifluoromethyl-biphenyl-2-carboxylic acid [4-(piperazinyl)-phenyl]-amide (318 mg) and 3-cyano-α-methyl-benzyl chloride (135 mg).

### EXAMPLE 3

4′-Isopropyl-6-methyl-biphenyl-2arboxylic acid [4-(4-(3-cyano-α-methyl-benzyl)-piperazin-1yl)-phenyl]-amide as a yellow powder (70 mg),

m.p.: 169-171° C.

Analysis: C36H38N4O1

Calc: C,79.67 ;H, 7.06 ;N,10.32;

Found: C,79.25 ;H, 6.99 ;N,10.18%.

from 4′-isopropyl6-methyl-biphenyl-2-carboxylic acid [4-(piperazinyl)-phenyl]-amide (207 mg) and 3-cyanoa-methyl-benzyl chloride (99 mg).

### EXAMPLE 4

4′-Trifluoromethyl-biphenyl-2-carboxylic acid [4-(4-(3-trifluoromethyl-α-methyl-benzyl)-piperazin-1-yl)-phenyl]-amide as white crystals (230 mg),

m.p.: 158-160° C.

Analysis: C33H29F6N3O1

Calc: C,66.33 ;H,4.89 ;N,7.03;

Found: C,65.96 ;H,4.78 ;N ,6.89%.

from 4′-trifluoromethyl-biphenyl-2-carboxylic acid [4-(piperazinyl)-phenyl]-amide (212 mg) and 3-trifluoromethyl-α-methyl-benzyl chloride (160 mg).

### EXAMPLE 5

4′-Trifluoromethyl-biphenyl-2-carboxylic acid [4-(4-(α-cyano-benzyl)-piperazin-1-yl)-phenyl]-amide as ecru crystals (0.83 g),

m.p.: 204° C.

MS: m/z 541 (M+1)

from 4′-trifluoromethyl-biphenyl-2-carboxylic acid [4-(piperazinyl)-phenyl]-amide (1 g) and α-cyano-benzyl bromide (0.51 g).

### EXAMPLE 6

4′-Trifluoromethyl-biphenyl-2-carboxylic acid [4-(4-(3-bromo-α-cyano-benzyl)-piperazin-1-yl)-phenyl]-amide as light yellow crystals (120 mg),

m.p.: 136° C.

MS: m/z 620 (M+1)

from 4′-trifluoromethyl-biphenyl-2-carboxylic acid [4-(piperazinyl)-phenyl]-amide (200 mg) and α-cyano-benzyl bromide (110 mg).

### EXAMPLE 7

4′-trifluoromethyl-biphenyl-2-carboxylic acid [4-(4-(4-fluoro-α-acetyl-benzyl)-piperazin-1-yl)-phenyl]-amide as white crystals (130 mg),

m.p.: 200-202° C.

MS: m/z 576 (M+1)

from 4′-trifluoromethyl-biphenyl-2-carboxylic acid [4-(piperazinyl)-phenyl]-amide (318 mg) and 4-fluoro-α-acetyl-benzyl bromide (190 mg).

### EXAMPLE 8

(GW 642690X) (FCBS/210/108/1)

4′-Trifluoromethyl-biphenyl-2-carboxylic acid [4-(4-(α-benzoyl-benzyl)-piperazin-1-yl)-phenyl]-amide as yellow crystals (325 mg),

m.p.: 165° C.

MS: m/z 620 (M+1)

from 4′-trifluoromethyl-biphenyl-2-carboxylic acid [4-(piperazinyl)-phenyl]-amide (300 mg) and α-benzoyl-benzyl bromide (200 mg).

### EXAMPLE 9

(GW 635028X) (FNDO/227/29/1)

4′-Trifluoromethyl-biphenyl-2-carboxylic acid [4-(4-(α(5-phenyl-[1,2,4]oxadiazol-3-yl)-benzyl)-piperazin-1-yl)-phenyl]-amide as ccru crystals (155 mg),

mp≈100° C.

MS: m/z 660 (M+1)

fom 4′-trifluoromethyl-biphenyl-2-carboxylic acid [4-(piperazinyl)-phenyl]-amide (250 mg) and α-(5-phenyl-[1,2,4]oxadiazol-3-yl)-benzyl bromide (204 mg).

### EXAMPLE 10

4′-Trifluoromethyl-biphenyl-2-carboxylic acid [4-(4-(α-((pyrazol-1-yl)-methyl)-benzyl)-piperazin-1-yl)-phenyl]-amide as White Crystals (200 mg),.

m.p.≈197° C.

MS: m/z 596 (M+1)

from 4′-trifluoromethyl-biphenyl-2-carboxylic acid [4-(piperazinyl)-phenyl]-amide (250 mg) and α-((pyrazol-1-yl)-methyl)-benzyl bromide (162 mg).

### EXAMPLE 11

4′-Trifluoromethyl-biphenyl-2-carboxylic acid [4-(4-(α-(methoxcyarbonyl)-benzyl)-piperazin-1-yl)-phenyl]-amide as white crystals (1.9 g),

m.p.: 130-135° C.

MS: m/z 574 (M+1)

from 4′-trifluoromethyl-biphenyl-2-carboxylic acid [4-(piperazinyl)-phenyl]-amide (1.6 g) and α-(methoxycarbonyl)-benzyl bromide (0.95 g).

### EXAMPLE 12

4′-Trifluoromethyl-biphenyl-2-carboxylic acid [4-(4-(α-(carboxy)-benzyl)-piperazin-1-yl)-phenyl]-!amide

To a suspension of 4′-trifluoromethyl-biphenyl-2-carboxylic acid [4-(4-(α-(methoxycarbonyl)-benzyl)-piperazin-1-yl)-phenyl]-amide (1.6 g) in methanol (50 mL) was added a solution of NaOH (1N, 11 mL) and the mixture was heated under reflux during 4 hours. After cooling, a solution of HCl (1N, 11 mL) was then added. After extraction with AcOEt, the organic phase was washed with brine dried over Na₂SO₄, filtered and concentrated under reduced pressure. The residue was triturated with diisopropyle oxyde, filtered and recrystallised from ethanol. The title compound was obtained as white crystals (1.3 g).

m.p.: 190-200° C.

MS: m/z 560 (M+1).

### EXAMPLE 13

4′-Trifluoromethyl-biphenyl-2-carboxylic acid [4-(4-(α-((2,2,2-trifluoroethyl)-aminocarbonyl)-benzyl)-piperazin-1-yl)-phenyl]-amide

To a stirred solution of 4′-trifluoromethyl-biphenyl-2-carboxylic acid [4-(4-(α-(carboxy)-benzyl)-piperazin-1-yl)-phenyl]-amide (330 mg), 2,2,2-trifluoroethylamine hydrochloride (86 mg), HOBT (94 mg) and triethylamine (139 mg) in CH₂Cl₂ (8 mL) was added EDCl (132 mg) and the mixture was stirred at room temperature overnight. The mixture was diluted with CH₂Cl₂, and the organic solution was washed with water, then with a saturated solution of NaHCO₃, then with a saturated solution of NaCl and dried over Na₂SO₄. After filtration and evaporation of the filtrate, the residue was purified by flash chromatography eluting with CH₂Cl₂/MeOH (97/3) . After crystallization from diisopropyle oxyde, the title compound was obtained as white crystals (210 mg).

m.p. : 206-208° C.

Analysis: C34H30F3N4O2

Calc: C,63.75 ;H,4.72 ;N,8.75;

Found: C,63.65 ;H,5.07 ;N,8.53%.

Similarly prepared were:

### EXAMPLE 14

4′-Trifluoromethyl-biphenyl-2-carboxylic acid [4-(4-(α-((pyridin-2-yl-methyl)-aminocarbonyl)-benzyl)-piperazin-1-yl)-phenyl]-amide as white crystals (210 mg),

m.p.: 139-141° C.

Analysis: C38H34F3N5O2

Calc: C,70.25 ;H,5.27 ;N,10.78;

Found: C,69.73 ;H,5.25 ;N,10.46%.

from 4-trifluoromethyl-biphenyl-2-carboxylic acid [4-(4-(α-(carboxy)-benzyl)-piperazin-1-yl)-phenyl]-amide (280 mg).

### EXAMPLE 15

4′-trifluoromethyl-biphenyl-2-carboxylic acid[4-(4-(3-bromo-α-thiocarbamoyl-benzyl)-piperazin-1-yl)-phenyl]-amide

A mixture of 4′-trifluoromethyl-biphenyl-2-carboxylic acid [4-(4-(3-bromo-α-cyano-benzyl)-piperazin-1-yl)-phenyl]-amide (0.8 g), diethyl-dithiophosphate (1.08 mL) and water (1 drop) was stirred at room temperature during 24 hours and then diluted with water. After extraction with CH₂Cl₂, the organic phase was washed with water, dried over Na₂SO₄, filtered and concentrated under reduced pressure. The residue was purified by flash chromatography, eluting with CH₂Cl₂/MeOH (95/5). The title compound was obtained as a yellow powder (400 mg).

m.p.: 135° C.

MS: m/z 654 (M+1).

### EXAMPLE 16

4′-Trifluoromethyl-biphenyl-2-carboxylic acid [4-(4-(3-bromo-α-(4-methyl-thiazol-2-yl)-benzyl)-piperazin-1 -yl)-phenyl]-amide

To a stirred solution of 4′-trifluoromethyl-biphenyl-2-carboxylic acid [4-(4-(3-bromo-α-thiocarbamoyl-benzyl)-piperazin-1-yl)-phenyl]-amide (350 mg) in ethanol (30 mL) was added chloroacetone (0.051 mL) and the mixture was heated under reflux overnight and then pourred into water. After extraction with CH₂Cl₂, the organic phase was dried over Na₂SO₄, filtered and concentrated under reduced pressure. The residue was purified by flash chromatography eluting with CH₂Cl_{2/}MeOH (95/5). The title compound was obtained as a cream powder (187 mg).

m.p.: 80° C

MS: m/z 692 (M+1).

### EXAMPLE 17

4′-Trifluoromethyl-biphenyl-2-carboxylic acid [4-(4-(3-cyano-α-(pyrrol-2-yl)-benzyl)-piperazin-1-yl)-phenyl]-amide

To a stirred solution of 4′-trifluoromethyl-biphenyl-2-carboxylic acid [4-(piperazinyl)-phenyl]-amide (360 mg) and 3-cyano-α-(pyrrol-2-yl)-benzyl alcool (250 mg) in THF (20 mL) were added diethyl azodicarboxylate (0.2 mL) and tributyl phosphine (0.315 mL) and the mixture was heated under reflux during 24 hours and then pourred into water. After extraction with CH₂Cl₂, the organic phase was dried over Na₂SO₄, filtered and concentrated under reduced pressure. The residue was purified by flash chromatography, eluting with CH₂C₂/MeOH (95/5). After crystallisation from AcOE/EtOH , the title compound was obtained as white crystals (50 mg).

m.p.: 202-203° C.

MS: m/z 604 (M−1).

Similarly prepared were:

### EXAMPLE 18

4′-Trifluoromethyl-biphenyl-2-carboxylic acid [4-(4-(-α-methyl-pyrrol-2-yl)-piperazin-1-yl)-phenyl]-amide as white crystals (550 mg),

m.p.: 150-152° C.

MS: m/z 519 (M+1)

fom 4′-trifluoromethyl-biphenyl-2-carboxylic acid [4-(piperazinyl)-phenyl]-amide (2 g).

Biological Assay

ApoB-100 Assay

Primary human hepatocytes were seeded at 50 000 cells/well in 96 well plates. After an overnight adhesion phase, cells were incubated with compounds for 8 hours in RPMI medium containing 1% FCS, 4 μg/ml insulin, 100 nM dexamethasone and 50 μCi/ml ³⁵S-methionine. Compounds were dissolved in DMSO and tested onto cells from 1 μM to 1.6 nM. Production of radiolabeled apoB-100 and apoA-1 (used as a selectivity control) was quantified by analysis of supernatants using SDS PAGE and exposure of gels onto Phosphorimager screens. Inhibition of apoB-100 and apoA-1 secretion by compounds was calculated taking untreated cells as controls, and IC₅₀ of each compound was determined on both apoproteins.

Biological Assay

The human MTP activity assay was established using SPA technology. Donor liposomes were prepared with 3H-triolein and phosphatidylcholine, while acceptor liposomes contained biotinylated phosphatidylethanolamine and phosphatidylcholine. The MTP-mediated 3H-triolein transfer onto acceptor liposomes was allowed by a 25 min incubation at 37° C., and quantified by the addition of streptavidin-SPA beads.

| | MTP |
|---|---|
| Example | (nM) |
| 2 | 1 |
| 8 | 40 |
| 9 | 5.6 |
| 10 | 157 |
| 11 | 3.2 |
| 14 | 3 |

Tablet Compositions

The following compositions A and B can be prepared by wet granulation of ingredients (a) to (c) and (a) to (d) with a solution of povidone, followed by addition of the magnesium stearate and compression.

Composition A

| | | mg/tablet | mg/tablet |
|---|---|---|---|
| (a) | Active ingredient | 250 | 250 |
| (b) | Lactose B.P. | 210 | 26 |
| (c) | Sodium Starch Glycollate | 20 | 12 |
| (d) | Povidone B.P. | 15 | 9 |
| (e) | Magnesium Stearate | 5 | 3 |
| | | 500 | 300 |

Composition B

| | | mg/tablet | mg/tablet |
|---|---|---|---|
| (a) | Active ingredient | 250 | 250 |
| (b) | Lactose 150 | 150 | — |
| (c) | Avicel PH 101 | 60 | 26 |
| (d) | Sodium Starch Glycollate | 20 | 12 |
| (e) | Povidone B.P. | 15 | 9 |
| (f) | Magnesium Stearate | 5 | 3 |
| | | 500 | 300 |

Composition C

| | mg/tablet |
|---|---|
| Active ingredient | 100 |
| Lactose | 200 |
| Starch | 50 |
| Povidone | 5 |
| Magnesium Stearate | 4 |
| | 359 |

The following compositions D and E can be prepared by direct compression of the admixed ingredients. The lactose used in composition E is of the direct compression type.

Composition D

| | mg/tablet |
|---|---|
| Active ingredient | 250 |
| Magnesium Stearate | 4 |
| Pregelatinised Starch NF15 | 146 |
| | 400 |

Composition E

| | mg/tablet |
|---|---|
| Active ingredient | 250 |
| Magnesium Stearate | 5 |
| Lactose | 145 |
| Avicel | 100 |
| | 500 |

Composition F (Controlled Release Composition)

| | | mg/tablet |
|---|---|---|
| (a) | Active ingredient | 500 |
| (b) | Hydroxypropylmethylcellulose | 112 |
| | (Methocel K4M Premium) | |
| (c) | Lactose B.P. | 53 |
| (d) | Povidone B.P.C. | 28 |
| (e) | Magnesium Stearate | 7 |
| | | 700 |

The composition can be prepared by wet granulation of ingredients (a) to (c) with a solution of povidone, followed by addition of the magnesium stearate and compression.

Composition G (Enteric-Coated Tablet)

Enteric-coated tablets of Composition C can be prepared by coating the tablets with 25 mg/tablet of an enteric polymer such as cellulose acetate phthalate, polyvinylacetate phthalate, hydroxypropylmethyl-cellulose phthalate, or anionic polymers of methacrylic acid and methacrylic acid methyl ester (Eudragit L). Except for Eudragit L, these polymers should also include 10% (by weight of the quantity of polymer used) of a plasticizer to prevent membrane cracking during application or on storage. Suitable plasticizers include diethyl phthalate, tributyl citrate and triacetin.

Composition H (Enteric-Coated Controlled Release Tablet)

Enteric-coated tablets of Composition F can be prepared by coating the tablets with 50 mg/tablet of an enteric polymer such as cellulose acetate phthalate, polyvinylacetate phthalate, hydroxypropylmethyl- cellulose phthalate, or anionic polymers of methacrylic acid and methacrylic acid methyl ester (Eudragit L). Except for Eudragit L, these polymers should also include 10% (by weight of the quantity of polymer used) of a plasticizer to prevent membrane cracking during application or on storage. Suitable plasticizers include diethyl phthalate, tributyl citrate and triacetin.

(ii) Capsule Compositions

Composition A

Capsules can be prepared by admixing the ingredients of Composition D above and filling two-part hard gelatin capsules with the resulting mixture. Composition B (infra) may be prepared in a similar manner.

Composition B

| | | mg/capsule |
|---|---|---|
| (a) | Active ingredient | 250 |
| (b) | Lactose B.P. | 143 |
| (c) | Sodium Starch Glycollate | 25 |
| (d) | Magnesium Stearate | 2 |
| | | 420 |

Composition C

| | | mg/capsule |
|---|---|---|
| (a) | Active ingredient | 250 |
| (b) | Macrogol 4000 BP | 350 |
| | | 600 |

Capsules can be prepared by melting the Macrogol 4000 BP, dispersing the active ingredient in the melt and filling two-part hard gelatin capsules therewith.

Composition D

| | mg/capsule |
|---|---|
| Active ingredient | 250 |
| Lecithin | 100 |
| Arachis Oil | 100 |
| | 450 |

Capsules can be prepared by dispersing the active ingredient in the lecithin and arachis oil and filling soft, elastic gelatin capsules with the dispersion.

Composition E (Controlled Release Capsule)

| | | mg/capsule |
|---|---|---|
| (a) | Active ingredient | 250 |
| (b) | Microcrystalline Cellulose | 125 |
| (c) | Lactose BP | 125 |
| (d) | Ethyl Cellulose | 13 |
| | | 513 |

The controlled release capsule composition can be prepared by extruding mixed ingredients (a) to (c) using an extruder, then spheronising and drying the extrudate. The dried pellets are coated with a release controlling membrane (d) and filled into two-part, hard gelatin capsules.

Composition F (Enteric Capsule)

| | | mg/capsule |
|---|---|---|
| (a) | Active ingredient | 250 |
| (b) | Microcrystalline Cellulose | 125 |
| (c) | Lactose BP | 125 |
| (d) | Cellulose Acetate Phthalate | 50 |
| (e) | Diethyl Phthalate | 5 |
| | | 555 |

The enteric capsule composition can be prepared by extruding mixed ingredients (a) to (c) using an extruder, then spheronising and drying the extrudate. The dried pellets are coated with an enteric membrane (d) containing a plasticizer (e) and filled into two-part, hard gelatin capsules.

Composition G (Enteric-Coated Controlled Release Capsule)

Enteric capsules of Composition E can be prepared by coating the controlled-release pellets with 50 mg/capsule of an enteric polymer such as cellulose acetate phthalate, polyvinylacetate phthalate, hydroxypropylmethylcellulose phthalate, or anionic polymers of methacrylic acid and methacrylic acid methyl ester (Eudragit L). Except for Eudragit L, these polymers should also include 10% (by weight of the quantity of polymer used) of a plasticizer to prevent membrane cracking during application or on storage. Suitable plasticizers include diethyl phthalate, tributyl citrate and triacetin.

(iii) Intravenous Injection Composition

| | |
|---|---|
| Active ingredient | 0.200 g |
| Sterile, pyrogen-free phosphate buffer (pH 9.0) to | 10 ml |

The active ingredient is dissolved in most of the phosphate buffer at 35-40° C., then made up to volume and filtered through a sterile micropore filter into sterile 10 ml glass vials (Type 1) which are sealed with sterile closures and overseals.

(iv) Intramuscular Injection Composition

| | |
|---|---|
| Active ingredient | 0.20 g |
| Benzyl Alcohol | 0.10 g |
| Glycofurol 75 | 1.45 g |
| Water for Injection q.s. to | 3.00 ml |

The active ingredient is dissolved in the glycofurol. The benzyl alcohol is then added and dissolved, and water added to 3 ml. The mixture is then filtered through a sterile micropore filter and sealed in sterile 3 ml glass vials (Type 1).

(v) Syrup Composition

| | |
|---|---|
| Active ingredient | 0.25 g |
| Sorbitol Solution | 1.50 g |
| Glycerol | 1.00 g |
| Sodium Benzoate | 0.005 g |
| Flavour | 0.0125 ml |
| Purified Water q.s. to | 5.0 ml |

The sodium benzoate is dissolved in a portion of the purified water and the sorbitol solution added. The active ingredient is added and dissolved. The resulting solution is mixed with the glycerol and then made up to the required volume with the purified water.

(vi) Suppository Composition

| | mg/suppository |
|---|---|
| Active ingredient | 250 |
| Hard Fat, BP (Witepsol H15 - Dynamit NoBel) | 1770 |
| | 2020 |

One-fifth of the Witepsol H15 is melted in a steam-jacketed pan at 45° C. maximum. The active ingredient is sifted through a 200 lm sieve and added to the molten base with mixing, using a Silverson fitted with a cutting head, until a smooth dispersion is achieved. Maintaining the mixture at 45° C., the remaining Witepsol H15 is added to the suspension which is stirred to ensure a homogenous mix. The entire suspension is then passed through a 250 lm stainless steel screen and, with continuous stirring, allowed to cool to 40° C. At a temperature of 38-40° C., 2.02 g aliquots of the mixture are filled into suitable plastic moulds and the suppositories allowed to cool to room temperature.

(vii) Pessary Composition

| | mg/pessary |
|---|---|
| Active ingredient (63 lm) | 250 |
| Anhydrous Dextrose | 380 |
| Potato Starch | 363 |
| Magnesium Stearate | 7 |
| | 1000 |

The above ingredients are mixed directly and pessaries prepared by compression of the resulting mixture.

(viii) Transdermal Composition

| | |
|---|---|
| Active ingredient | 200 mg |
| Alcohol USP | 0.1 ml |
| Hydroxyethyl cellulose | |

The active ingredient and alcohol USP are gelled with hydroxyethyl cellulose and packed in a transdermal device with a surface area of 10 cm².

1. A compound of formula (I); 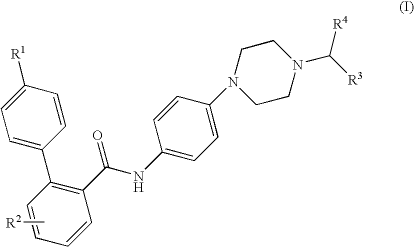
wherein
R¹ represents isopropyl or trifluoromethyl;
R² represents hydrogen, C₁₋₄alkyl, chloro, fluoro or trifluoromethyl;
R³ represents
(i) phenyl, optionally substituted by cyano, halogen, trifluoromethyl or an optionally substituted 5-membered heteroaromatic group, where optional substitution is effected by C₁₋₄alkyl,
(ii) a 5-membered heteroaromatic group, optionally substituted by halogen, cyano or C₁₋₄alkyl,
(iii) aminocarbonyl, or
(iv) ethyl or eth-1-enyl;
R⁴ represents cyano, methyl, acetyl, a 5-membered heteroaromatic group, optionally substituted by C₁₋₄alkyl or phenyl, or a group X—Y-Z;
X represents a carboxy, oxo, C₁₋₆alkylene, carboxamido or thiocarboxamido linking group;
Y represents a direct link or C₁₋₆alkylene;
Z represents
(i) hydrogen,
(ii) trifluoromethyl,
(iii) cyano,
(iv) phenyl,
(v) a 5- or 6-membered heteroaromatic group, optionally substituted by C₁₋₄alkyl,
with the proviso that when X represents C₁₋₆alkylene, Y and Z do not represent a direct link and hydrogen respectively, or when X represents oxo, Y and Z do not represent C₁₋₆alkylene and hydrogen respectively;
or a physiologically acceptable salt, solvate or derivative thereof.

2. A compound according to claim 1 where R¹ is isopropyl.

3. A compound according to claim 1 or 2 where R² is methyl or isopropyl and is 5- or 6-substituted.

4. A compound according to any one of claims 1-3 where R³ is selected from phenyl, optionally substituted by cyano, trifluoromethyl or halogen, or a 5-membered heteroaromatic group.

5. A compound according to any one of claims 1-4 where R⁴ represents
(i) cyano,
(ii) benzoyl,
(iii) hydroxycarbonyl, C₁₋₄alkoxycarbonyl, e.g. methoxycarbonyl, C₁₋₃perfluoroalkylaminocarbonyl, e.g. 1,1,1-trifluoroethylaminocarbonyl,
(iv) aminothiocarbonyl;
(v) a 5-membered heteroaromatic group, e.g. oxadiazolyl or pyrrolyl, optionally substituted by phenyl, or
(vi) a 5-membered heteroaromatic group linked by a methylene, e.g. pyrazolylmethyl.

6. A compound according to claim 1 represented by a compound of formula (Ia) 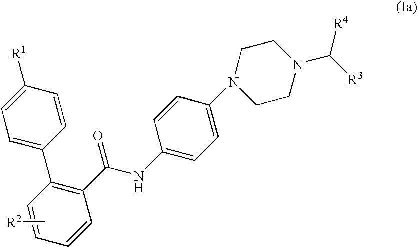
wherein
R¹ represents isopropyl or trifluoromethyl;
R² represents hydrogen, C₁₋₄alkyl, chloro, fluoro or trifluoromethyl;
R³ represents
(i) phenyl, optionally substituted by cyano, halogen, trifluoromethyl or an optionally substituted 5-membered heteroaromatic group, where optional substitution is effected by C₁₋₄alkyl, or
(ii) a 5-membered heteroaromatic group, optionally substituted by halogen, cyano or C₁₋₄alkyl;
R⁴ represents cyano, a 5-membered heteroaromatic group, optionally substituted by C₁₋₄alkyl or phenyl, or a group X—Y-Z;
X represents a carboxy, oxo, C₁₋₆alkylene, carboxamido or thiocarboxamido linking group;
Y represents a direct link or C₁₋₆alkylene;
Z represents
(i) hydrogen,
(ii) trifluoromethyl,
(iii) cyano,
(iv) phenyl,
(v) a 5- or 6-membered heteroaromatic group, optionally substituted by C₁₋₄alkyl,
with the proviso that when X represents C₁₋₆alkylene, Y and Z do not represent a direct link and hydrogen respectively, or when X represents oxo, Y and Z do not represent C₁₋₆alkylene and hydrogen respectively;
or a physiologically acceptable salt, solvate or derivative thereof.

7. A compound according to claim 1 which is selected from:
4′-trifluoromethyl-biphenyl-2-carboxylic acid[4-(4-(α-methyl-benzyl)-piperazin-1-yl)-phenyl]-amide;
5-methyl-4′-trifluoromethyl-biphenyl-2-carboxylic acid[4-(4-(3-cyano-α-methyl-benzyl)-piperazin-1-yl)-phenyl]-amide;
4′-isopropyl-6-methyl-biphenyl-2-carboxylic acid[4-(4-(3-cyano-α-methyl-benzyl)-piperazin-1-yl)-phenyl]-amide;
4′-trifluoromethyl-biphenyl-2-carboxylic acid[4-(4-(3-trifluoromethyl-α-methyl-benzyl)-piperazin-1 -yl)-phenyl]-amide;
4′-trifluoromethyl-biphenyl-2-carboxylic acid[(4-(α-cyano-benzyl)-piperazin-1-yl)-phenyl]-amide;
4′-trifluoromethyl-biphenyl-2-carboxylic acid[4-(4-(3-bromo-α-cyano-benzyl)-piperazin-1-yl)-phenyl]-amide;
4′-trifluoromethyl-biphenyl-2-carboxylic acid[4-(4-(4-fluoro-α-acetyl-benzyl)-piperazin-1-yl)-phenyl]-amide;
4′-trifluoromethyl-biphenyl-2-carboxylic acid[4-(4-(α-benzoyl-benzyl)-piperazin-1-yl)-phenyl]-amide;
4′-trifluoromethyl-biphenyl-2-carboxylic acid[4-(4-(α-(5-phenyl-[1,2,4]oxadiazol-3-yl)-benzyl)-piperazin-1-yl)-phenyl]-amide;
4′-trifluoromethyl-biphenyl-2-carboxylic acid[4-(4-(α-((pyrazol-1-yl)-methyl-benzyl)-piperazin-1-yl)-phenyl]-amide;
4′-trifluoromethyl-biphenyl-2-carboxylic acid[4-(4-(α-(methoxycarbonyl)-benzyl)-piperazin-1-yl)-phenyl)-amide;
4′-trifluoromethyl-biphenyl-2-carboxylic acid[4-(4-(α-(carboxy)-benzyl)-piperazin-1-yl)-phenyl]-amide;
4′-trifluoromethyl-biphenyl-2-carboxylic acid[4-(4-(α-((2,2,2-trifluoroethyl)-aminocarbonyl)-benzyl)-piperazin-1-yl)-phenyl]-amide;
4′-trifluoromethyl-biphenyl-2-carboxylic acid[4-(4-(α-((pyridin-2-yl-methyl)-aminocarbonyl)-benzyl)-piperazin-1-yl)-phenyl]-amide;
4′-trifluoromethyl-biphenyl-2-carboxylic acid[4-(4-(3-bromo-α-thiocarbamoyl-benzyl)-piperazin-1-yl)-phenyl]-amide;
4′-trifluoromethyl-biphenyl-2-carboxylic acid[4-(4-(3-bromo-α-(4-methyl-thiazol-2-yl)-benzyl)-piperazin-1-yl)-phenyl]-amide;
4′-trifluoromethyl-biphenyl-2-carboxylic acid[4-(4-(3-cyano-α-(pyrrol-2-yl)-benzyl)-piperazin-1-yl)-phenyl]-amide;
4′-trifluoromethyl-biphenyl-2-carboxylic acid[4(4-(α-methyl-pyrrol-2-yl)-piperazin-1-yl)-phenyl]-amide;
or a physiologically acceptable salt, solvate or derivative thereof.

8. A compound according to any one of claims 1 to 7 for use in therapy.

9. A method for the treatment of a mammal, including man, of conditions ameliorated by an apoB-100 and/or MTP inhibitor comprising administration of an effective amount of a compound according to any one of claims 1 to 8 or a pharmaceutically acceptable derivative thereof.

10. The use of a compound according to any one of claims 1 to 8 or a physiologically acceptable salt or solvate thereof in the manufacture of a medicament for use in the treatment of conditions ameliorated by an apoB-100 and/or MTP inhibitor.

11. A pharmaceutical composition comprising a compound according to any one of claims 1 to 8 or a pharmaceutically acceptable derivative thereof together with one or more pharmaceutically acceptable carriers.

12. A process for the preparation of a compound of formula (I) comprising:
(A) reacting a compound of formula (II) with a compound of formula R³(R⁴)L 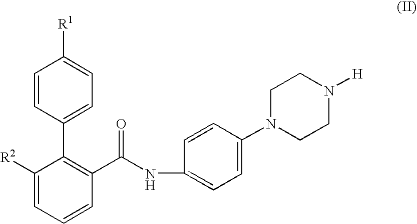
where L represents a suitable leaving group or a hydroxy group;
(B) reaction of compounds of formula (III) and compounds of formula (VI) 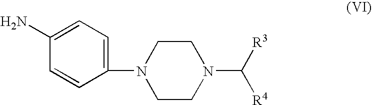
where L is defined above;
(C) where there is an alkylene link to the piperidine or piperazine group, reacting a compound of formula (II) with a compound of formula (VII); 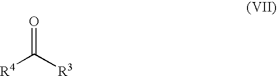
(D) by reaction of a different compound of formula (I), using standard techniques well known in the art.
